(19) United States
(12) Patent Application Publication
(10) Pub. No.: US 2013/0289473 A1
(43) Pub. Date: Oct. 31, 2013

(54) Medical Conduit

(71) Applicant: Tak Suen Sze, Hong Kong (CN)

(72) Inventor: Tak Suen Sze, Hong Kong (CN)

(21) Appl. No.: 13/565,817

(22) Filed: Aug. 3, 2012

(30) Foreign Application Priority Data
Apr. 30, 2012 (HK) 12104257.6

(51) Int. Cl. A61M 25/14

(52) U.S. Cl. 604/26; 604/246

(57) ABSTRACT

A medical conduit includes a first conduit, a second conduit and a third conduit. The first conduit is bigger than the third conduit in length and built within the third conduit; one end of the first conduit penetrates a bore at one end of the third conduit, while the other end of the first conduit penetrates a through-hole on the side wall of the other end of the third conduit and, together with the other end of the third conduit, forms a Y shape; the side wall at the other end of the first conduit is provided with a number of through-holes for guiding liquid or gas; one end of the second conduit is embedded at the bore at one end of the third conduit. The medical conduit can perform guidance and location in a human body and separately conduct injection and exhaustion to provide accurate measuring data for doctors.

### BACKGROUND OF THE PRESENT INVENTION

1. Field of Invention

The present invention relates to a medical instrument, in particular to a medical conduit.

2. Description of Related Arts

During operations, a doctor needs to insert a conduit into the esophagus or trachea of a patient (even deep into the stomach or lung) from the nasal or oral cavity to guide in the required drugs (such as anesthetic gas) or deliver oxygen gas.

The prior art provides a conduit. One end of the conduit is connected with an inlet-outlet valve so as to be adapted to a medical instrument. The other end of the conduit is inserted into a patient's body from the oral or nasal cavity.

The oral and nasal cavities of humans are connected, so no matter whether the conduit is inserted from the oral cavity or nasal cavity, the conduit will enter the larynx, which leads to the esophagus (deep into the stomach) or the trachea (deep into the lung).

In the prior art, there is only straight conduit inserted into the larynx, so it is difficult for a doctor to control the insertion of the conduit into the esophagus or the trachea.

In the modern anesthetic practice, measurement of the carbon dioxide exhausted by the patient during operation can provide impotent information about the patient. A conduit design to undertake oxygen delivery and exhaled gas analysis can facilitate the judgment of the doctor and reduce risks to the patient.

### SUMMARY OF THE PRESENT INVENTION

To solve the above mentioned technical problem, the present invention provides a medical conduit.

The medical conduit comprises:

a first conduit, a second conduit and a third conduit; wherein

the first conduit is bigger than the third conduit in length and smaller in outer diameter and is built within the third conduit; one end of the first conduit penetrates a bore at one end of the third conduit and is connected with a first inlet-outlet valve, while the other end of the first conduit penetrates a through-hole on a side wall of the other end of the third conduit and, together with the other end of the third conduit, forms a Y shape; a side wall at the other end of the first conduit is provided with a number of through-holes for guiding liquid or gas;

one end of the second conduit is embedded at the bore at one end of the third conduit, while the other end of the second conduit is connected with a second inlet-outlet valve; also, the second conduit is communicated with the third conduit.

The bore at one end of the third conduit is sealed with a glue material.

The present invention provides a medical conduit which can be accurately inserted into the esophagus or trachea to perform drug delivery and exhalation gas analysis at the same time, providing accurate measuring data for the doctors and helping the doctor accurately judge the patient's condition.

### BRIEF DESCRIPTION OF THE DRAWINGS

To describe the technical scheme in the embodiment of the present invention or in the prior art more clearly, the following are brief introductions to the attached drawings used in the description. Obviously, the attached drawings only illustrate some embodiments of the present invention. For those skilled in this field, other drawings can be obtained according to the attached drawings without creative labor.

FIG. 1 is a structural view of a medical conduit provided by the present invention;

FIG. 2 is an application schematic view of a medical conduit provided by the present invention;

FIG. 3 is another application schematic view of a medical conduit provided by the present invention;

FIG. 4 is another application schematic view of a medical conduit provided by the present invention;

FIG. 5 is another application schematic view of a medical conduit provided by the present invention.

### DETAILED DESCRIPTION OF THE PREFERRED EMBODIMENT

The present invention provides a medical conduit which can be located in a human body to be accurately inserted into the esophagus or trachea and perform drug delivery and exhalation gas analysis at the same time, providing accurate measuring data for the doctors and helping the doctor accurately judge the patient's condition.

The following are detailed descriptions of the medical conduit provided by the present invention.

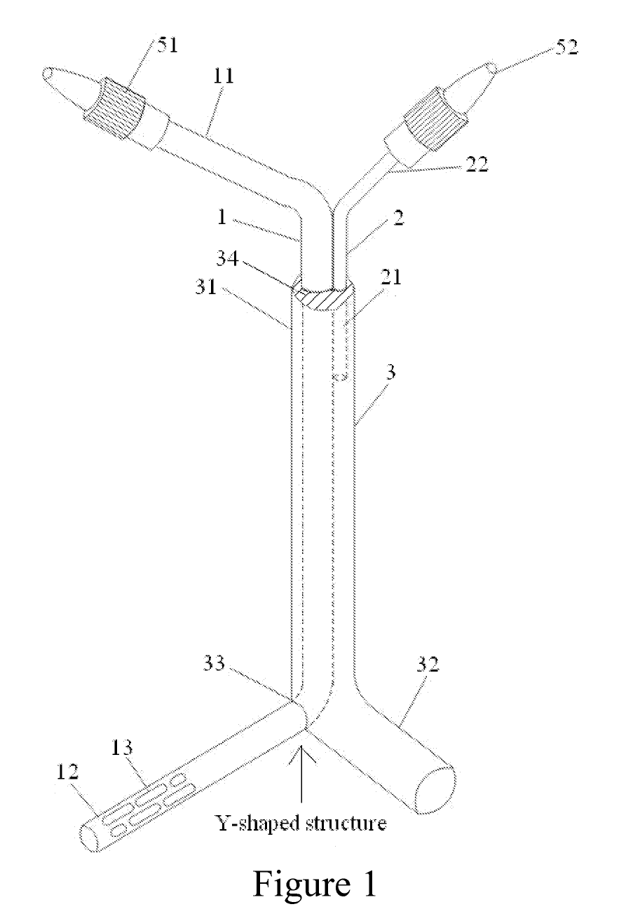

FIG. 1 is a structural view of a medical conduit in embodiment 1 provided by the present invention.

Embodiment 1 provides a medical conduit which comprises:

a conduit 1, a conduit 2 and a conduit 3; wherein

conduit 1 is bigger than conduit 3 in length and smaller in outer diameter and is built within conduit 3; the first end 11 of conduit 1 penetrates a bore at one end 31 of conduit 3 and is connected with a first inlet-outlet valve 51, while the second end 12 of conduit 1 penetrates a through-hole 33 on the side wall of the second end 32 of conduit 3 and, together with the second end 32 of conduit 3, forms a Y shape 4; the side wall at the second end 12 of conduit 1 is provided with a number of through-holes 13 for guiding liquid or gas;

the first end 21 of conduit 2 is embedded at the bore at the first end 31 of conduit 3, and the second end 22 of conduit 2 is connected with an inlet-outlet valve 52; also, conduit 2 is communicated with conduit 3.

The bore at the first end 31 of the third conduit 3 is sealed with a glue material 34. The glue material is usually flexible glue to avoid other liquids or gases seeping into conduit 3.

In this embodiment, the second end 12 of conduit 1 and the second end 32 of conduit 3 are formed into Y shape 4, which helps a doctor control the direction to insert conduit 1 into the esophagus or trachea. The inlet-outlet valve 51 connected with the first end 11 of conduit 1 is adapted to the medical instrument for inputting liquid or gas into conduit 1; the liquid or gas flows into the esophagus located by the doctor along with the through-hole 13 formed on the side wall at the second end 12 of conduit 1, or the gas flows into the trachea located by the doctor. Besides, the carbon dioxide generated by the patient passes through the bore at the end of the 32 of conduit 3 and then is discharged from conduit 2; the second end 22 of conduit 2 is connected with the inlet-outlet valve 52, so the doctor can measure the content of the carbon dioxide discharged from the patient by adapting a medical instrument to the inlet-outlet valve 52 to judge the patient's condition.

The following are the details of the description of the medical conduit of the present invention with the references of body views.

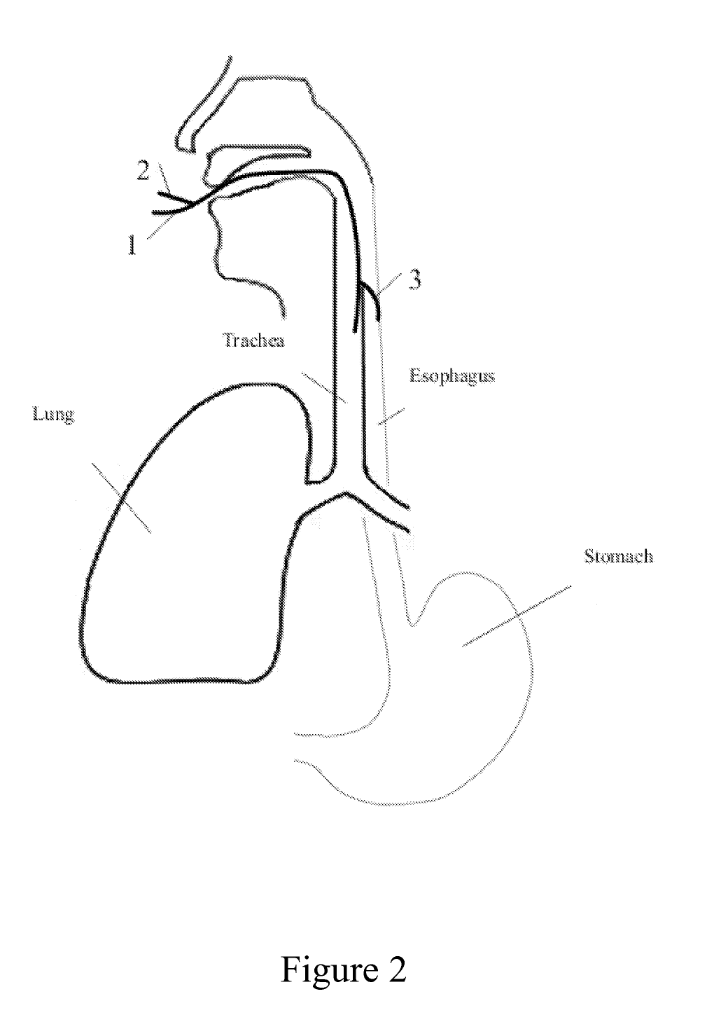

As shown in FIG. 2, when a doctor inserts the medical conduit provided by the present invention into a patient's larynx, the Y-shaped structure 4 will be stopped at the supraglottic area, where the larynx branch into the esophagus and trachea.

The doctor can insert the medical conduit to the expected position (esophagus or trachea) with simple maneuver, first by introducing conduit 1 to the expected position.

After the conduit 1 has entered into either esophagus or trachea, simply turn or twist the conduit by 180°, conduit 3 will then follow the direction of conduit 1.

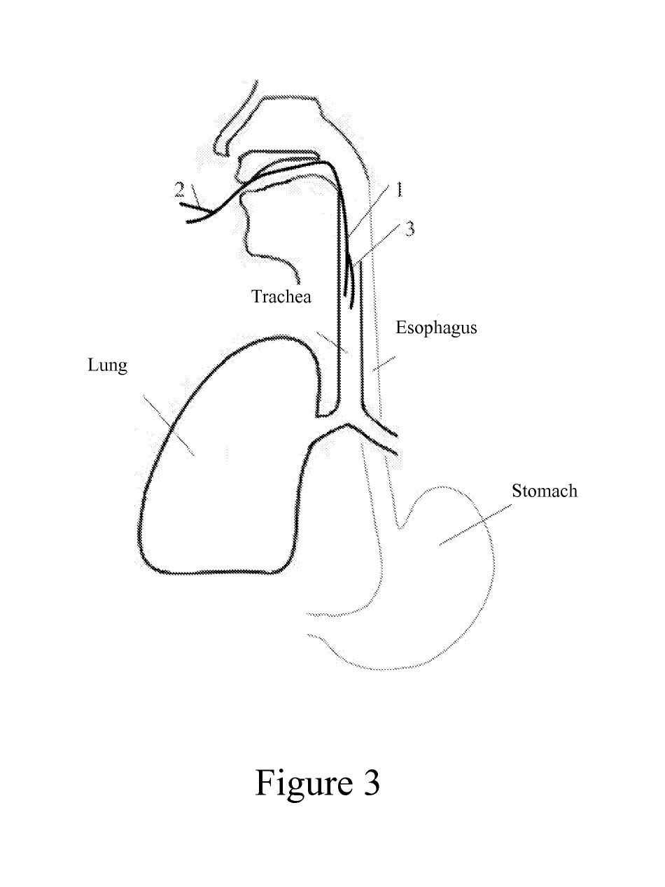

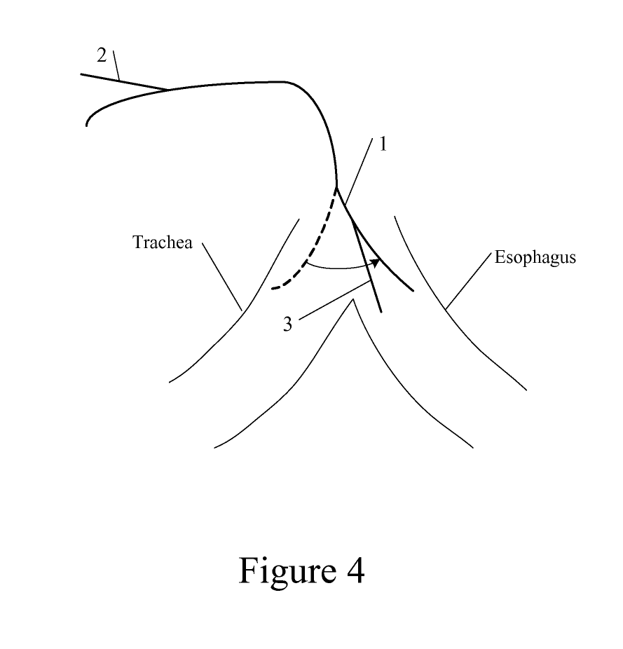

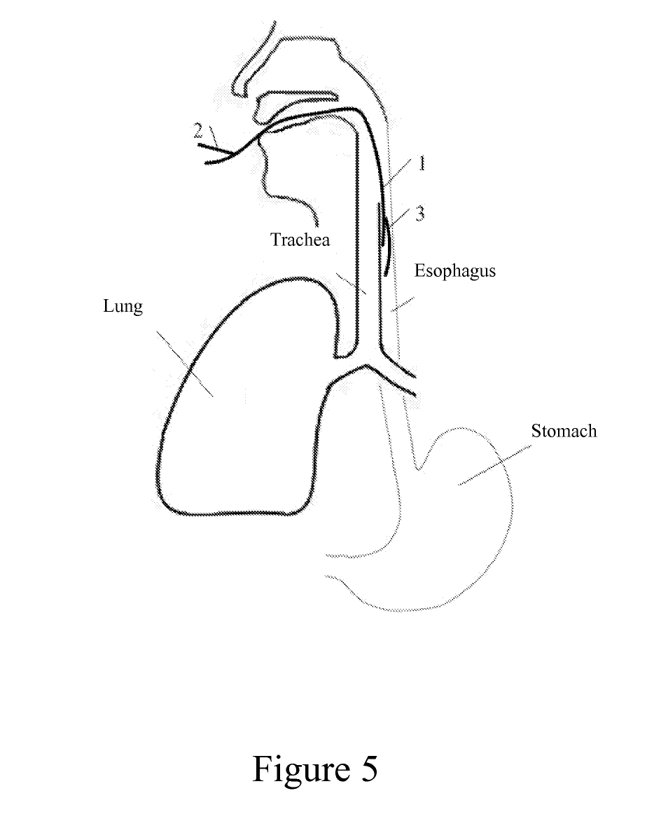

As shown in FIG. 3, the doctor slightly moves conduit 3 to insert conduit 1 and conduit 3 into the trachea to realize pulmonary drug delivery. On the contrary, if the doctor intends to insert conduit 1 and conduit 3 into the esophagus, as shown in FIG. 4, the doctor only needs to rotate conduit 1 of the medical conduit at an angle of 180° from the position aligned with the trachea (shown by the virtual line) to the position aligned with the esophagus. As shown in FIG. 5, at this moment, the doctor can insert conduit 1 and conduit 3 into the esophagus to realize gastric drug delivery.

Many ducts in the human body have several branches, so the lower end of the medical conduit provided by this present invention also can be shaped into multi-branch structure as applicable to the body structure and can be set to input gas and liquid via several branches and output gas from the patient via one branch.

Thus, the medical conduit provided by the present invention is applicable to various ducts in the human body besides the esophagus and trachea.

It should be noted that the medical conduit described in the present invention is just a product form, so other products with the structure of the present invention are within the protective scope of the present invention even if factors such as the texture, instrument title, appearance and placement sequence without influences on the product features are different.

The above contents further describe the present invention with the references of the preferable embodiments, which shall not be regarded as limitations of the present invention. For those skilled in this field, many simple deductions or substitutions can be made on the premise of the concept of the present invention, which all are within the protective scope of the present invention.

What is claimed is:

1. A medical conduit, comprising:
a first conduit, a second conduit and a third conduit; wherein
one end of the first conduit penetrates a bore at one end of the third conduit and is connected with a first inlet-outlet valve, while the other end of the first conduit penetrates a through-hole on a side wall of the other end of the third conduit and, together with the other end of the third conduit, forms a Y shape; a side wall at the other end of the first conduit is provided with a number of through-holes for guiding liquid or gas;
one end of the second conduit is embedded at the bore at one end of the third conduit, while the other end of the second conduit is connected with a second inlet-outlet valve.

2. The medical conduit according to claim 1, characterized in that the bore at one end of the third conduit is sealed by a glue material.

3. The medical conduit according to claim 1, characterized in that the first conduit is bigger than the third conduit in length and smaller in outer diameter and is built within the third conduit.

4. The medical conduit according to claim 2, characterized in that the first conduit is bigger than the third conduit in length and smaller in outer diameter and is built within the third conduit.

5. The medical conduit according to claim 1, characterized in that the second conduit is communicated with the third conduit.

6. The medical conduit according to claim 2, characterized in that the second conduit is communicated with the third conduit.

7. The medical conduit according to claim 3, characterized in that the second conduit is communicated with the third conduit.

8. The medical conduit according to claim 4, characterized in that the second conduit is communicated with the third conduit.
